(19) United States
(12) Patent Application Publication
(10) Pub. No.: US 2026/0240741 A1
(43) Pub. Date: Aug. 20, 2026

(54) COMPOSITION USEFUL FOR SLOWING DOWN DEGRADATION CAUSED BY LIGHT, HEAT AND/OR THE OXIDATION OF AN ACTIVE COMPOUND

(71) Applicant: SENSIENT COSMETIC TECHNOLOGIES, SAINT-OUEN L'AUMONE (FR)

(72) Inventor: Anthony DEGOURNAY, ULLY-SAINT-GEORGES (FR)

(21) Appl. No.: 19/472,551

(22) PCT Filed: Apr. 4, 2024
(86) PCT No.: PCT/EP2024/059124

§ 371 Date: Oct. 3, 2025

(30) Foreign Application Priority Data
Apr. 6, 2023 (FR) FR2303441

(51) Int. Cl. A61K 8/49; A61K 8/37; A61Q 13/00; A61Q 19/00

(52) U.S. Cl. CPC A61K 8/4973; A61K 8/37; A61Q 13/00; A61Q 19/00

(57) ABSTRACT

The present invention relates to a composition comprising:
a compound A of formula (I):

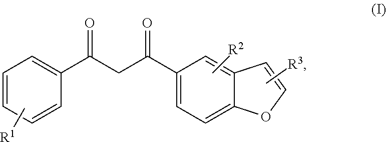

and
a compound B of formula (II):

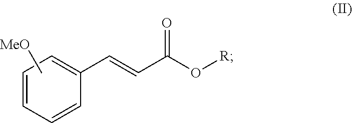

to the use thereof for slowing, or even preventing, de gradation caused by light, heat and/or the oxidation of at least one active compound selected from photosensitive, heat-sensitive and/or oxidation-sensitive compounds; and to a formulation comprising the composition.

The present invention relates to compositions that allow the stabilization of photosensitive, heat-sensitive, or more broadly, oxidation-sensitive active compounds in formulations, notably cosmetic, dermo-cosmetic, pharmaceutical, and/or household, industrial, and institutional detergent products (“Household, Industrial & Institutional (HI&I) products”).

In the fields of cosmetics, dermo-cosmetics, pharmacy, and industry, many formulations comprise one or more active compound(s), typically chosen from dyes, odorant molecules, UV filters, antioxidants, and vitamins. This active compound, of natural or synthetic origin, is generally intended to improve the appearance, sensory qualities, or functionality of the formulations.

For aesthetic reasons, such formulations are often packaged in transparent packaging, for example, glass or plastic materials, but these packages allow visible light and ultraviolet (UV) rays to pass through.

Active compounds sensitive to light, particularly ultraviolet rays, are then likely to degrade, which can alter the appearance and/or properties of the formulations.

Exposure to ultraviolet rays can indeed induce radical reactions, modifying the chemical structure of photosensitive compounds, such as dyes or odorant molecules in a perfume. This results in a change in the color and/or odor of the formulations, or even a loss of activity of the active compounds.

A common solution to improve the stability of formulations against ultraviolet rays is the use of UV filters.

As ultraviolet filters, compounds from the benzophenone family or other compounds absorbing ultraviolet rays are notably used, such as those mentioned, for example, in Annex VI of the cosmetic regulation (EC) No. 1223/2009, in Annex VII of the Directive C.E.E. No. 76/768 (relating to ultraviolet filters accepted in cosmetics), or in the guidelines of the F.D.A. (American Food and Drugs Administration), namely in the Federal Register, vol. 43, no. 166 (Aug. 25, 1978).

However, benzophenones have revealed a number of drawbacks, notably a strong yellow coloration upon introduction and sometimes insufficient protection, and are toxic to a number of marine organisms and classified as carcinogenic, mutagenic, and reprotoxic (CMR).

More generally, organic UV filters are materials derived from petrochemicals and are increasingly controversial as they are suspected of having endocrine-disrupting activity, as is the case for ethylhexyl salicylate.

However, these compounds are intended to protect the skin and hair against the harmful effects of UV rays, and not the formulations themselves or their photosensitive compounds, such as odorant molecules and dyes. One can refer to the article “Filtres et écrans solaires” by M. C. Poelman in “Actifs et additifs en cosmétologie,” 2nd edition, 1999, Editions Tec & Doc, Paris. The use of a single UV filter in a formulation does not always ensure sufficiently effective protection of dyes against ultraviolet rays.

EP 2 985 018 thus describes compositions allowing the stabilization of a coloration of a formulation against UV radiation. These compositions consist of the combination of three ultraviolet filters belonging respectively to the cinnamates, dibenzoylmethanes, and salicylates families. In the examples, compositions consisting of ethylhexyl methoxycinnamate, butyl methoxydibenzoylmethane, and ethylhexyl salicylate were used. However, ethylhexyl salicylate is suspected of being an endocrine disruptor by European regulatory bodies. Moreover, this application remains silent on the ability of the composition to protect other cosmetic actives than dyes.

Other factors can cause the degradation of active compounds, notably heat and oxidation.

FR 3 023 715 relates to a synergistic combination of at least two agents promoting the photoprotective efficacy of at least one sunscreen filter, one of the two said agents being a promoter of the photoprotective efficacy of the sunscreen filter(s) against UVA and being chosen from flavonoids and diarylheptanoids, the other of the two said agents being a promoter of the photoprotective efficacy of the sunscreen filter(s) against UVB and being chosen from phenolic acids and their derivatives. In the examples, combinations of pongamol and ferulic acid, and a combination of pongamol, ferulic acid, and para-coumaric acid are used.

However, the effects of these combinations are limited to their ability to protect sunscreen filters, and there is no mention in this application of any ability of the combinations to protect other cosmetic actives.

The development of alternative stabilizing compositions limiting the degradation of at least one active compound of a formulation by light, heat, and/or oxidation is required.

One of the objectives of the present invention is therefore to propose a composition to stabilize the aforementioned type of formulations against the harmful effects of light, heat, and/or oxidation and being compatible with the active compound(s) present in these formulations.

In this context, the invention particularly aims to provide compositions allowing the stabilization of at least one cosmetic, dermo-cosmetic, pharmaceutical, or detergent active, this active being a photosensitive, heat-sensitive, and/or oxidation-sensitive compound, and generally chosen from odorant molecules, dyes, vitamins, antioxidants, UV filters, and mixtures thereof.

To this end, the invention relates to a composition comprising:
(A) a compound A of formula (I):

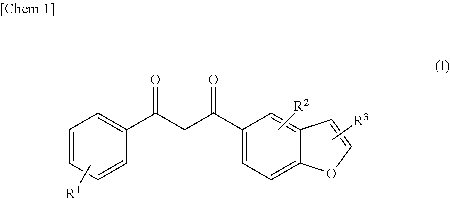

wherein R¹, R², and R³ independently represent H, a hydroxy group, a C₁-C₄ alkyl group, a C₁-C₄ alkoxy group; and
(B) a compound B of formula (II):

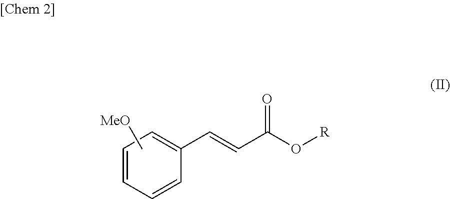

wherein R represents a C₁-C₄ alkyl group.

Compound A is useful as an ultraviolet filter against UV-A radiation. Compound B is useful as an ultraviolet filter against UV-B radiation.

In the context of this description, “ultraviolet filter” (or “UV filter”) means a chemical compound capable of absorbing electromagnetic radiation within a range included in the ultraviolet radiation domain, namely in the wavelength range between 10 and 400 nanometers. Notably, ultraviolet UV-A radiation, with a wavelength between 315 and 400 nanometers, and ultraviolet UV-B radiation, with a wavelength between 280 and 315 nanometers, are distinguished.

The invention also relates to the use of the aforementioned type of composition to slow down, or even prevent, degradation caused by light, heat, and/or oxidation of at least one active compound chosen from photosensitive, heat-sensitive, and/or oxidation-sensitive compounds in a formulation, the active compound being preferably chosen from odorant molecules, dyes, vitamins, antioxidants, UV filters, and mixtures thereof.

The invention relates to the use of the composition defined above to protect at least one such active compound against degradation caused by light, heat, and/or oxidation.

The invention also relates to a formulation comprising 0.05 to 5.00% by weight of the aforementioned type of composition, and at least one active compound chosen from photosensitive, heat-sensitive, and/or oxidation-sensitive compounds, preferably chosen from odorant molecules, dyes, vitamins, antioxidants, UV filters, and mixtures thereof.

The formulation according to the invention can be a cosmetic, dermo-cosmetic, pharmaceutical, or household, industrial, and institutional detergent product formulation (“Household, Industrial & Institutional (HI&I) products”).

The inventors have found that the combination of compounds A and B ensures very effective stabilization of the active compounds in a formulation against light, notably ultraviolet radiation. The inventors have discovered that, surprisingly, the combination of compounds A and B is suitable for reducing the degradation of at least one active compound generally used in cosmetic, dermo-cosmetic, pharmaceutical, and/or household, industrial, and institutional detergent product formulations (“Household, Industrial & Institutional (HI&I) products”). The active compound is chosen from photosensitive, heat-sensitive, and/or oxidation-sensitive compounds, preferably chosen from odorant molecules, dyes, vitamins, antioxidants, UV filters, and mixtures thereof, preferably dyes, odorant molecules, and mixtures thereof, particularly preferably dyes.

Compound A, useful as a UV-A filter, belongs to the dibenzoylmethane family. It matches formula (I):

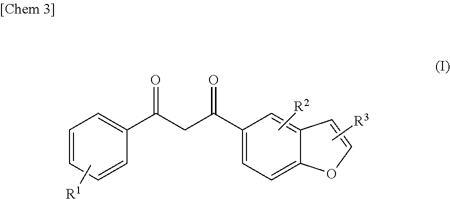

wherein R¹, R², and R³ independently represent H, a hydroxy group, a C₁-C₄ alkyl group, and a C₁-C₄ alkoxy group. Preferably, R¹, R², and R³ independently represent H, OH, Me, or OMe.

In particular, compound A matches formula (Ia):

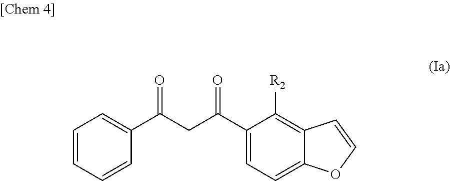

In a particularly advantageous way, compound A is pongamol of formula (Ib):

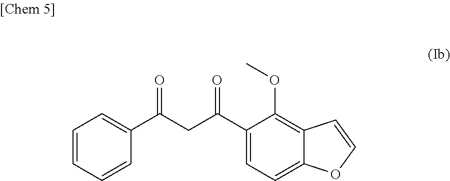 [SMILES: COc1c(C(=O)CC(=O)c2ccccc2)ccc2occc12]

Furanoflavonoids, and more particularly pongamol, are derived from plants of the legume family, notably from species such as Pongamia pinnata, Pongamia glabra, Millettia pinnata, Derris indica, Gadelupa pinnata, Pongamia grandifolia, Robinia mitis, Tephrosia purpurea, Tephrosia hamiltoni, Tephrosia falciformis, Tephrosia vogellii, Tephrosia lanceolata, etc. Within these species, pongamol is primarily present in the seed, and more specifically in the oil of these seeds, where it generally represents about 27 to 39% by weight relative to the weight of the oil. It can be obtained by mechanical or chemical extraction. Pongamol can be in the form of karanja oil, which is typically extracted from the seed, or it can also be produced by synthetic chemistry.

Preferably, compound A is in the form of a Pongamia pinnata seed extract, with a pongamol weight concentration preferably greater than 70%, and more specifically greater than 95%, relative to the weight of the extract.

Compound B, useful as a UV-B ultraviolet filter, belongs to the cinnamate derivatives family. It matches formula (II):

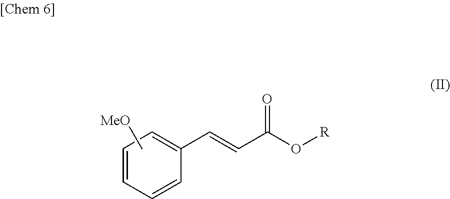

wherein R represents a C₁-C₄ alkyl group. Preferably, R represents a C₁-C₃alkyl group, particularly Et.

Preferably, compound B matches formula (IIa):

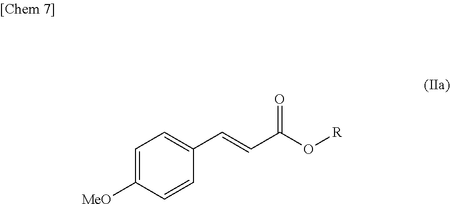

In a particularly advantageous way, compound B is ethyl-p-methoxycinnamate of formula (IIb):

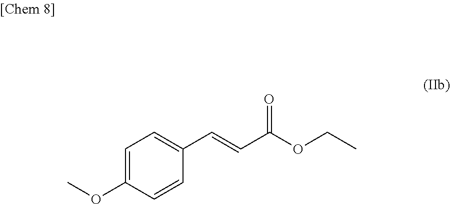

Ethyl-p-methoxycinnamate is a compound that can be of natural origin. Indeed, it is naturally present in plants of the Zingiberaceae family, more commonly used as spices or condiments, as well as medicinal herbs in Southeast Asia. This is notably the case for species such as Kaempferia galanga and Alpinia galanga, whose essential oil from the rhizome contains 0.1% to 30.0% ethyl-p-methoxycinnamate.

Preferably, ethyl-p-methoxycinnamate is in the form of a Kaempferia galanga root extract, with a weight concentration of ethyl-p-methoxycinnamate preferably greater than 70%, and more specifically greater than 98%.

The composition comprises compounds A and B in proportions that can vary widely.

However, a preferred composition comprises:
5 to 95% by weight of compound A; and
5 to 95% by weight of compound B;
relative to the total weight of compounds A and B.

This composition is particularly effective for protecting at least one active compound in a formulation against degradation due to light, notably UV radiation, and/or heat and/or oxidation.

Particularly preferred are compositions that comprise:
20 to 95% by weight of compound A; and
5 to 80% by weight of compound B;
relative to the total weight of compounds A and B.

Advantageously, the composition comprises:
30 to 85% by weight of compound A; and
15 to 70% by weight of compound B;
relative to the total weight of compounds A and B.

According to a preferred embodiment, the composition comprises:
45 to 75% by weight of compound A; and
25 to 55% by weight of compound B;
relative to the total weight of compounds A and B.

Thus, according to a particular embodiment, the composition comprises:
50 to 70% by weight of compound A; and
30 to 50% by weight of compound B;
relative to the total weight of compounds A and B.

Generally, the sum of the proportions of compounds A and B represents 20% to 100% by weight, notably 50 to 100% by weight, preferably 70 to 100% by weight, relative to the weight of the composition.

Compounds A and B are soluble in most organic solvents, particularly in alcohols or esters. They can also be solubilized in the active compound to be stabilized or in a formulation comprising the active compound to be stabilized.

It may be desirable to introduce the composition into an aqueous formulation or one comprising a hydrophilic phase, particularly an aqueous phase (typically an emulsion). For this purpose, the composition may comprise one or more ionic or non-ionic surfactants. A surfactant with an HLB greater than or equal to 12 is preferred as it facilitates the solubilization of compounds A and B in an aqueous phase.

The composition may also comprise a solubilizing agent, which facilitates the total or partial dissolution of compounds A and B and allows a composition in liquid or paste form to be obtained. This solubilizing agent is preferably a compound or a mixture of compounds commonly used in cosmetic, dermo-cosmetic, pharmaceutical, or industrial applications. Typically, the solubilizing agent is an organic solvent, for example, an alcohol or an ester.

Depending on the nature, purity, origin, and proportion of compounds A and B and the nature and proportion of any additional compounds (solubilizing agent, surfactant, etc.), the composition can be in solid or liquid form, allowing the form of the composition to be adapted according to the intended application.

In one embodiment, the composition consists of:
compounds A and B,
and optionally a surfactant, a solubilizing agent, or a mixture thereof.

In its simplest form, the composition consists of compounds A and B.

In a preferred embodiment, the composition comprises (or consists of) pongamol and ethyl-p-methoxycinnamate, and optionally a surfactant, a solubilizing agent, or a mixture thereof.

The composition may notably comprise (or consist of):
a Pongamia pinnata seed extract that comprises pongamol, typically at least 27% by weight, preferably at least 70% by weight of pongamol relative to the weight of the Pongamia pinnata seed extract,
a Kaempferia galanga root extract that comprises ethyl-p-methoxycinnamate, typically at least 0.1% by weight, preferably at least 70% by weight of ethyl-p-methoxycinnamate relative to the weight of the Kaempferia galanga root extract.

According to a second aspect, the invention relates to the use of the composition defined above to slow down, or even prevent, degradation caused by light, heat, and/or oxidation of at least one active compound chosen from photosensitive, heat-sensitive, and/or oxidation-sensitive compounds in a formulation, the active compound being preferably chosen from odorant molecules, dyes, vitamins, antioxidants, UV filters, and mixtures thereof, particularly chosen from dyes, odorant molecules, and mixtures thereof.

The invention also relates to the use of the composition defined above to protect at least one such active compound against degradation caused by light, heat, and/or oxidation.

Among photosensitive and heat-sensitive active compounds, dyes can be mentioned, particularly dyes authorized in Annex IV of the cosmetic regulation, and preferably, the most used in fine perfumery and rinse-off products, comprising FD&C Yellow 5, FD&C Yellow 6, FD&C Yellow 10, FD&C Red 4, D&C Red 33, FD&C Red 40, FD&C Orange 4, Ext D&C Violet 2, FD&C Blue 1, and any other synthetic dye generally used in targeted applications and mixtures thereof.

Dyes can be of natural origin, such as carmine, caramel, annatto, comprising bixin and norbixin, turmeric, comprising curcuminoids (curcumin, demethoxycurcumin, and bisdemethoxycurcumin), spirulina and its phycobiliproteins, anthocyanidins and their heterosides (anthocyanins), betalains (including betacyanins, such as betanin, and betaxanthins, such as indicaxanthin), carotenoids (comprising beta-carotene, capsanthin, and capsorubin), free or complexed chlorophyll and chlorophyllin, xanthophylls (comprising astaxanthin and lutein), or any other natural molecule with coloring properties.

The active compound can be an odorant molecule.

The present invention particularly relates to the use of the composition defined above to stabilize the odorant molecules of a formulation, preferably a perfume formulation, from the harmful effects of visible light, ultraviolet rays, temperature, or any other oxidation mechanism, and thus prevent its degradation.

For example, odorant molecules belonging to the family of esters (comprising geranyl acetate, isoamyl acetate, pentyl butyrate, or ethyl butyrate), linear terpenes (comprising geraniol, myrcene, citral, or linalool) and cyclic terpenes (comprising limonene, menthol, camphor, or eucalyptol), aromatics (comprising eugenol, vanillin, anisole, or benzaldehyde), amines (comprising pyridine, indole, or cadaverine), alcohols (comprising furaneol or 1-hexanol), aldehydes (comprising acetaldehyde, furfural, or hexyl cinnamaldehyde), ketones (comprising dihydrojasmone or cyclopentadecanone), lactones (comprising jasmine lactone, sotolon, or gamma-decalactone), thiols (comprising furan-2-ylmethanethiol, allyl-thiol, or thioacetone), essential oils, for example, those of bergamot, eucalyptus, orange, lemon, lavender, geranium, almond, or a mixture thereof.

The active compound can be a vitamin or an antioxidant (for example, ascorbic acid, tocopherol, retinol, niacinamide, or their derivatives) and/or a UV filter (such as avobenzone, octocrylene, ethylhexyl salicylate, ethylhexyl triazone, bis-ethylhexyloxyphenol methoxyphenyl triazine, phenylbenzimidazole sulfonic acid, or diethylhexyl butamido triazone).

The invention further relates to a method for slowing down, or even preventing, degradation caused by light, heat, and/or oxidation of at least one active compound chosen from photosensitive, heat-sensitive, and/or oxidation-sensitive compounds comprising contacting the active compound (or a formulation, notably cosmetic, comprising such an active compound) with a composition as defined above.

According to a third aspect, the invention relates to a formulation comprising 0.05% to 5.00% by weight of the aforementioned type of composition, and at least one active compound chosen from photosensitive, heat-sensitive, and/or oxidation-sensitive compounds, preferably chosen from odorant molecules, dyes, vitamins, antioxidants, UV filters, and mixtures thereof.

The formulation can be a cosmetic, dermo-cosmetic, pharmaceutical, or household, industrial, and institutional product formulation.

In particular, the formulation may comprise from 0.05% to 1.00%, especially from 0.05% to 0.30%, preferably from 0.10% to 0.20%, for example 0.15% by weight of the aforementioned type of composition relative to the total weight of the formulation.

The formulation can be a hydrophilic formulation, for example, aqueous, alcoholic, or hydro-alcoholic, or an oily formulation. It can be monophasic or biphasic. For example, it can be in the form of an emulsion and comprise an oily phase and a hydrophilic phase, particularly an aqueous phase. The formulation can be a cream, gel, serum, mist, or lotion.

Advantageously, the formulation comprises at least 10% alcohol, preferably ethanol, relative to the weight of the formulation. In this case, the composition can be solubilized within the formulation, which then forms a single homogeneous phase as is the case for hydro-alcoholic formulations, such as perfumes, eau de parfum, eau de toilette, and eau de Cologne. The presence of a surfactant within the composition or the emulsion is then not required.

When the formulation comprises an oily phase, the presence of a surfactant facilitates the dispersion of the composition within the formulation. The composition typically appears as microdroplets and forms a dispersed phase within the formulation. In this case, the composition preferably comprises a surfactant and/or the formulation preferably comprises a surfactant. The surfactant is notably as defined above.

The formulation can be a perfume (comprising at least 10% alcohol, or less than 10% alcohol, for example, an alcohol-free perfume) or a care and hygiene product for the face, body, and/or hair.

The formulation is preferably a perfume.

Other advantages and features of the present invention will become apparent from the illustrative examples presented below.

### Example 1: Preparation of Compositions and Formulations

The tested compositions combine a UV A absorbing material, which is either avobenzone (or butyl methoxydibenzoylmethane) (comparative), or a Pongamia pinnata seed extract (here named “Pongamia extract,” or compound A), and a UV B absorbing material, which is either OMC (or ethylhexyl methoxycinnamate) (comparative), or a Kaempferia galanga root extract (here named “galanga extract” or compound B). These compositions are listed in Table 1, which provides the weight ratio of compound(s) within the composition, said composition being solely comprised of this/these compound(s) (no solvent or surfactant added).

For the compound Avobenzone, Eusolex 9010 (Merck) (avobenzone proportion greater than 95%) or Parsol 1789 (DSM) (avobenzone proportion greater than 95%) were used. For the Pongamia extract, Pongamol (Indfrag Biosciences) (pongamol proportion greater than 95%) was used.

For the Galanga extract, Galanga Extract (Sabinsa) (ethyl-p-methoxycinnamate proportion greater than 98%) was used.

TABLE 1
Stabilizing compositions

| | |
|---|---|
| Composition | Ratio |

| | | | |
|---|---|---|---|
| 01 | No protection | — | Reference |
| 02 | Avobenzone | — | Reference |
| 03 | Pongamia extract | — | Reference |
| 04 | Avobenzone:OMC | 10:1 | Comparative |
| 05 | Avobenzone:Galanga extract | 10:1 | Comparative |
| 06 | Pongamia extract:Galanga extract | 10:1 | Invention |
| 07 | Avobenzone:OMC | 5:1 | Comparative |
| 08 | Avobenzone:Galanga extract | 5:1 | Comparative |
| 09 | Pongamia extract:Galanga extract | 5:1 | Invention |
| 10 | Avobenzone:OMC | 5:2 | Comparative |
| 11 | Avobenzone:Galanga extract | 5:2 | Comparative |
| 12 | Pongamia extract:Galanga extract | 5:2 | Invention |
| 13 | Avobenzone:OMC | 2:1 | Comparative |
| 14 | Avobenzone:Galanga extract | 2:1 | Comparative |
| 15 | Pongamia extract:Galanga extract | 2:1 | Invention |
| 16 | Avobenzone:OMC | 1:1 | Comparative |
| 17 | Avobenzone:Galanga extract | 1:1 | Comparative |
| 18 | Pongamia extract:Galanga extract | 1:1 | Invention |
| 19 | Avobenzone:OMC | 1:2 | Comparative |
| 20 | Avobenzone:Galanga extract | 1:2 | Comparative |
| 21 | Pongamia extract:Galanga extract | 1:2 | Invention |
| 22 | Avobenzone:OMC | 2:5 | Comparative |
| 23 | Avobenzone:Galanga extract | 2:5 | Comparative |
| 24 | Pongamia extract:Galanga extract | 2:5 | Invention |
| 25 | Avobenzone:OMC | 1:5 | Comparative |
| 26 | Avobenzone:Galanga extract | 1:5 | Comparative |
| 27 | Pongamia extract:Galanga extract | 1:5 | Invention |
| 28 | OMC | — | Reference |
| 29 | Galanga extract | — | Reference |

To evaluate and demonstrate their performance, 0.15% by weight of one of the compositions 1 to 29 were added to different formulations. These formulations had the composition detailed in Table 2, with proportions being by weight relative to the weight of the formulation.

TABLE 2
Perfume formulations comprising compositions 1 to 29

| Formulas | F1 | F2 | F3 |
|---|---|---|---|
| Perfume | Oriental Infinity | Oriental Infinity | Diamond Beauty |
| | 8% | 8% | 8% |
| Dye | FD&C Yellow 5 | FD&C Red 33 | FD&C Red 33 |
| (solution at 0.1%) | 1% | 1% | 1% |
| Compositions 1 to 29 | 0.15% | 0.15% | 0.15% |
| Ethanol | 75% | 75% | 75% |
| Water | 15.85% | 15.85% | 15.85% |

The formulations F1, F2, and F3 were characterized using a transmission spectrocolorimeter, model CM-5 (Konica Minolta), according to the L*a*b CIE 1976 standard, or CIELAB 1976, using the D65 colorimetric standard where:
L* represents luminance or lightness: this axis ranges from the value 0, which is the absence of light (black), to the value 100, which is full light (white);
The value a* represents the axis from green (−100) to red (+100);
The value b* represents the axis from blue (−100) to yellow (+100).

The formulations are then placed in an accelerated UV aging chamber (Suntest) for 24 hours, and their color is measured again to evaluate the difference between the sample before and after UV stability testing. The ΔE values are then calculated using the following formula:

$$Δ {E}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

where L*₁, a*₁, and b*₁ are the colorimetric coordinates of the control sample before suntest, and L*₂, a*₂, and b*₂ are the colorimetric coordinates of the sample after suntest.

### Example 2: Stabilization Tests of Perfume Formulation F1

TABLE 3
Performance of the compositions on the stabilization of F1

| | | % |
|---|---|---|
| Composition | ΔE* | efficacy |
| 01 | 32.22 | 0.0% |
| 02 | 22.48 | 30.2% |
| 03 | 16.63 | 48.4% |
| 04 | 21.66 | 32.8% |
| 05 | 19.77 | 38.6% |
| 06 | 14.61 | 54.6% |
| 07 | 19.64 | 39.1% |
| 08 | 17.83 | 44.7% |
| 09 | 10.53 | 67.3% |
| 10 | 21.02 | 34.8% |
| 11 | 17.20 | 46.6% |
| 12 | 11.57 | 64.1% |
| 13 | 21.42 | 33.5% |
| 14 | 16.92 | 47.5% |
| 15 | 12.86 | 60.1% |
| 16 | 24.14 | 25.1% |
| 17 | 21.01 | 34.8% |
| 18 | 12.81 | 60.2% |
| 19 | 26.98 | 16.3% |
| 20 | 20.23 | 37.2% |
| 21 | 17.98 | 44.2% |
| 22 | 28.66 | 11.1% |
| 23 | 20.70 | 35.8% |
| 24 | 20.41 | 36.7% |
| 25 | 31.33 | 2.8% |
| 26 | 27.86 | 13.5% |
| 27 | 24.11 | 25.2% |
| 28 | 33.58 | −4.2% |
| 29 | 33.22 | −3.1% |

The results provided in Table 3 demonstrate that the F1 formulations comprising extracts enriched with compounds A and extracts enriched with compounds B are more stable than the F1 formulations comprising the UV filters Avobenzone/OMC, or Avobenzone/Compound B, regardless of the ratio, from 10:1 (compound demonstrating anti-UV-A properties: compound demonstrating anti-UV-B properties) to 1:5.

Within the compositions according to the invention comprising compounds A and B, compositions 06, 09, 12, 15, and 18 demonstrate the most interesting color protection performances: respectively 54.6%, 67.3%, 64.1%, 60.1%, and 60.2%. These performances are superior to those of compounds A and B separately, which are 48.4% and −3.1%, respectively.

### Exemple 3: Stabilization Tests of the Perfume Formulation F2

TABLE 4
Performance of the compositions on the stabilization of F2

| | | % |
|---|---|---|
| Composition | ΔE* | efficacity |
| 01 | 46.37 | 0.0% |
| 02 | 30.40 | 34.4% |
| 03 | 29.98 | 35.3% |
| 04 | 30.97 | 33.2% |
| 05 | 29.87 | 35.6% |
| 06 | 27.72 | 40.2% |
| 07 | 31.30 | 32.5% |
| 08 | 29.87 | 35.6% |
| 09 | 27.18 | 41.4% |
| 10 | 31.53 | 32.0% |
| 11 | 28.16 | 39.3% |
| 12 | 26.16 | 43.6% |
| 13 | 32.08 | 30.8% |
| 14 | 27.34 | 41.0% |
| 15 | 26.45 | 43.0% |
| 16 | 32.04 | 30.9% |
| 17 | 29.79 | 35.8% |
| 18 | 26.50 | 42.8% |
| 19 | 31.61 | 31.8% |
| 20 | 26.63 | 42.6% |
| 21 | 26.56 | 42.7% |
| 22 | 37.48 | 19.2% |
| 23 | 31.97 | 31.1% |
| 24 | 25.76 | 44.4% |
| 25 | 41.55 | 10.4% |
| 26 | 33.08 | 28.7% |
| 27 | 29.48 | 36.4% |
| 28 | 45.68 | 1.5% |
| 29 | 46.03 | 0.7% |

The results provided in Table 4 demonstrate that the F2 formulations, identical to F1 except for the use of a different dye (FD&C Red 33), comprising extracts enriched with compound A and extracts enriched with compound B are more stable than the F2 formulations comprising the UV filters Avobenzone/OMC, or Avobenzone/Compound B, regardless of the ratio, from 10:1 (compound demonstrating anti-UV-A properties: compound demonstrating anti-UV-B properties) to 1:5.

Among the preferred compositions comprising compounds A and B, compositions 06, 09, 12, 15, 18, 21, 24, and 27 demonstrate interesting color protection performances: respectively 40.2%, 41.4%, 43.6%, 43.0%, 42.8%, 42.7%, 44.4%, and 36.4%. These performances are superior to those of compounds A and B separately, which are 35.3% and 0.7%, respectively.

### Example 4: Stabilization Tests of the Perfume Formulation F3

TABLE 5
Performance of the compositions on the stabilization of F3

| Composition | ΔE* | % efficacity |
|---|---|---|
| 01 | 46.52 | 0.0% |
| 02 | 24.65 | 47.0% |
| 03 | 20.53 | 55.9% |
| 04 | 23.86 | 48.7% |
| 05 | 23.48 | 49.5% |
| 06 | 18.93 | 59.3% |
| 07 | 23.45 | 49.6% |
| 08 | 22.74 | 51.1% |
| 09 | 18.14 | 61.0% |
| 10 | 22.88 | 50.8% |
| 11 | 22.30 | 52.1% |
| 12 | 18.16 | 61.0% |
| 13 | 23.80 | 48.8% |
| 14 | 22.86 | 50.8% |
| 15 | 18.39 | 60.5% |
| 16 | 24.92 | 46.4% |
| 17 | 24.58 | 47.2% |
| 18 | 19.27 | 58.6% |
| 19 | 26.38 | 43.3% |
| 20 | 25.75 | 44.6% |
| 21 | 20.09 | 56.8% |
| 22 | 28.68 | 38.3% |
| 23 | 27.39 | 41.1% |
| 24 | 20.40 | 56.1% |
| 25 | 33.56 | 27.9% |
| 26 | 32.16 | 30.9% |
| 27 | 26.92 | 42.1% |
| 28 | 44.46 | 4.4% |
| 29 | 44.92 | 3.4% |

The results provided in Table 5 demonstrate that the F3 formulations, identical to F2 except for the use of a different fragrance (Diamond Beauty), comprising extracts enriched with compound A and extracts enriched with compound B are more stable than the F3 formulations comprising the UV filters Avobenzone/OMC, or Avobenzone/Compound B, regardless of the ratio, from 10:1 (compound demonstrating anti-UV-A properties: compound demonstrating anti-UV-B properties) to 1:5.

Among the preferred compositions comprising compounds A and B, compositions 06, 09, 12, 15, 18, 21, and 24 demonstrate interesting color protection performances: respectively 59.3%, 61.0%, 61.0%, 60.5%, 58.6%, 56.8%, and 56.1%. These performances are superior to those of compounds A and B separately, which are 55.9% and 3.4%, respectively.

1. A composition comprising:
(A) A compound A of formula (I): 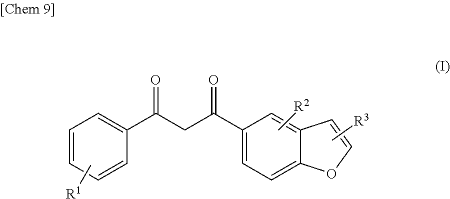
wherein R¹, R², and R³ independently represent H, a hydroxy group, a C₁-C₄ alkyl group,
or a C₁-C₄ alkoxy group; and
(B) A compound B of formula (II): 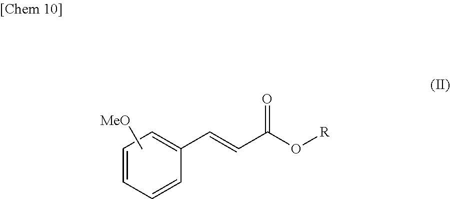
wherein R represents a C₁-C₄ alkyl group.

2. The composition according to claim 1, wherein R¹, R², and R³ independently represent H, −OH, Me, or OMe.

3. The composition according to claim 1, wherein compound A matches formula (Ia): 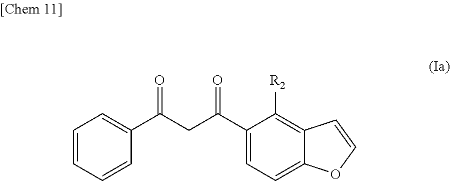
wherein R² is as defined in claim 1.

4. The composition according to claim 2, wherein compound A is pongamol of formula (Ib):

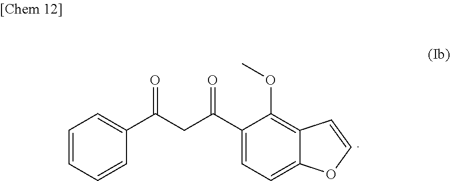 [SMILES: COc1c(C(=O)CC(=O)c2ccccc2)ccc2occc12]

5. The composition according to claim 1, wherein R represents a C₁-C₃ alkyl group.

6. The composition according to claim 1, wherein compound B matches formula (IIa): 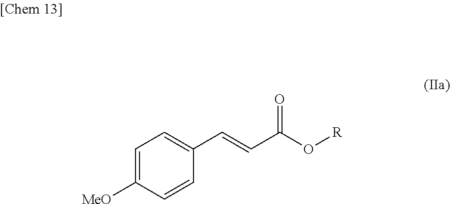
wherein R is as defined in claim 1.

7. The composition according to claim 6, wherein compound B is ethyl-p-methoxycinnamate of formula (IIb):

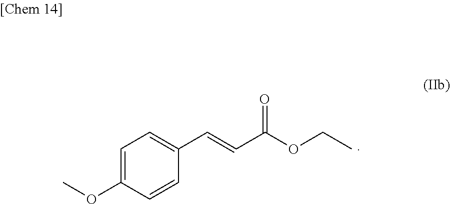

8. The composition according to claim 1, comprising:
from 5% to 95% by weight of compound A; and
from 5% to 95% by weight of compound B,
relative to the total weight of compounds A and B.

9. A method for slowing down, or even preventing, degradation caused by light, heat, and/or oxidation of at least one active compound selected from photosensitive, heat-sensitive, and/or oxidation-sensitive compounds, comprising contacting the active compound with the composition according to claim 1.

10. A formulation comprising from 0.05 to 5.00% by weight of the composition according to claim 1 and at least one active compound selected from photosensitive, heat-sensitive, and/or oxidation-sensitive compounds.

11. The formulation according to claim 10, which is a cosmetic, dermo-cosmetic, pharmaceutical, or household, industrial, and institutional product formulation.
